(19) United States
(12) Patent Application Publication
(10) Pub. No.: US 2026/0026675 A1
(43) Pub. Date: Jan. 29, 2026

(54) ENDOSCOPIC SYSTEMS AND METHODS WITH LARGE FIELD OF VIEW AND ARTIFICIAL INTELLIGENCE SUPPORT

(71) Applicant: VERILY LIFE SCIENCES LLC, Dallas, TX (US)

(72) Inventors: Roman Goldenberg, Haifa (IL); Itamar Talmi, Haifa (IL)

(21) Appl. No.: 19/144,298

(22) PCT Filed: Jan. 2, 2024
(86) PCT No.: PCT/US2024/010024

§ 371 Date: Jun. 27, 2025

Related U.S. Application Data
(60) Provisional application No. 63/478,253, filed on Jan. 3, 2023.

(51) Int. Cl. A61B 1/00; A61B 1/04; A61B 1/31

(52) U.S. Cl. CPC A61B 1/000096; A61B 1/0005; A61B 1/00055; A61B 1/04; A61B 1/31

(57) ABSTRACT

Methods and systems of analyzing and displaying medical videos are described herein. In some embodiments, the methods and systems may be used during an endoscopy procedure using a multi-camera endoscope having a first camera and a second camera. A method according to the present disclosure may include: receiving a first medical video from a first camera; receiving a second medical video from a second camera; detecting one or more target features of the second medical video using a pre-trained target feature detector comprising a neural network; generating an alert indicating the detection of the one or more target features in the second medical video; and displaying the first medical video and the alert on a display.

### CROSS-REFERENCES TO RELATED APPLICATIONS

The present application claims the benefit of and priority to U.S. Provisional Patent Application No. 63/478,253, filed Jan. 3, 2023, the entirety of which is hereby incorporated by reference. The present application is a national phase application of Patent Cooperation Treaty (PCT) Application No. PCT/US2024/010024, filed on Jan. 2, 2024, the entirety of which is hereby incorporated by reference.

### TECHNICAL FIELD

The present disclosure relates generally to endoscopic systems having at least one camera for an increased field of view and that use artificial intelligence to process video captured during endoscopy procedures and to methods of training and using such systems.

### BACKGROUND

Millions of Americans undergo colonoscopies per year, primarily as a prophylaxis against colon cancer. Generally, the physician performing the colonoscopy uses an endoscope to visually examine and identify polyps, cancer, or other potential problems in the colon. If the doctor identifies a polyp, she may decide to remove all or part of the polyp as a precaution. However, most endoscopes currently used contain only one camera with a standard angle, making it difficult for physicians to examine the entire colon, particularly behind the folds of the colon or in areas that are generally difficult to view by manually manipulating the camera over a large viewing angle.

In order to address this problem, the endoscope may have multiple standard angle cameras or one or more wide-angle cameras to increase the view during a procedure. However, using multiple cameras may require physicians to look at multiple images at once, which can be challenging and ineffective. Moreover, images collected from wide-angle cameras may be distorted along the edges of the images, making it difficult to accurately identify polyps in the expanded view. Thus, improved endoscopic systems are needed that can image the entire colon without adding a significant burden to the physician or providing misleading or inaccurate information.

### SUMMARY

Methods of analyzing and displaying medical videos are described herein. In some embodiments, methods may be performed during an endoscopy procedure using a multi-camera endoscope having a first camera and a second camera. The method may include: receiving a first medical video from a first camera; receiving a second medical video from a second camera; detecting one or more target features of the second medical video using a pre-trained target feature detector comprising a neural network; generating an alert indicating the detection of the one or more target features in the second medical video; and displaying the first medical video and the alert on a display.

In some embodiments, the endoscopy procedure may be a colonoscopy and the one or more target features may include a polyp. In some embodiments, the first medical video may have a first field of view and the second medical video may have a second field of view. In some embodiments, the first field of view may be different than the second field of view. In some embodiments, the method may also include determining a location of the second field of view relative to the first field of view. In some embodiments, detecting one or more target features may include detecting a location of the one or more target features in the second field of view. In some embodiments, the alert may indicate a location of the one or more target features relative to the first field of view. In some embodiments, the alert may include a part of the second medical video including the one or more target features. In some embodiments, the alert may include a box encompassing one or more of the one or more target features. In some embodiments, the first camera and the second camera may be coupled to an endoscope. In some embodiments, the target feature detector may include a domain-specific pre-trained machine learning model.

Systems for analyzing and displaying medical videos during an endoscopy procedure are described herein. The system may include an endoscope having a first camera and a second camera, a display, and a processor. The processor may be configured to: receive a first medical video from the first camera and receive a second medical video from the second camera. The processor may be further configured to detect one or more target features of the second medical video, e.g., using a target feature detector. The processor may be further configured to generate an alert indicating the detection of the one or more target features in the second medical video, and display the first medical video and the alert on the display.

In some embodiments, the first medical video may have a first field of view and the second medical video may have a second field of view. In some embodiments, the first field of view may be different than the second field of view. In some embodiments, the processor may be further configured to determine a location of the second field of view relative to the first field of view. In some embodiments, the processor is further configured to detect a location of the one or more target features in the second field of view. In some embodiments, the alert may indicate a location of the one or more target features relative to the first field of view.

Systems for analyzing and displaying medical videos during an endoscopy procedure are described herein. The system may include a first camera configured to receive a first medical video comprising a first field of view, a second camera configured to receive a second medical video comprising a second field of view different from the first field of view, a display configured to display at least the first medical video, and a processor. The processor may be configured to receive the second medical video; detect a target feature of the second medical video using a pre-trained target feature detector comprising a neural network; generate an alert indicating the detection of the target feature in the second medical video; and display the alert on the display.

In some embodiments, the alert may be displayed adjacent to the first medical video. In some embodiments, the alert may include a part of the second medical video comprising the target feature.

### BRIEF DESCRIPTION OF THE DRAWINGS

Illustrative embodiments of the present disclosure will be described with reference to the accompanying drawings, of which:

FIG. 1 is a simplified schematic diagram illustrating a computer system for implementing a target feature detector and alert generator, according to some embodiments of the present disclosure.

FIG. 2 is a simplified diagram of a multi-camera endoscope, according to some embodiments of the present disclosure.

FIG. 3 is a simplified diagram of a fisheye camera endoscope, according to some embodiments of the present disclosure.

FIG. 4 is a simplified flow chart illustrating use of the target feature detector and alert generator, according to some embodiments of the present disclosure.

FIG. 5 is a simplified diagram of a display, according to some embodiments of the present disclosure.

FIG. 6 is a simplified diagram of a display, according to some embodiments of the present disclosure.

FIG. 7 is a flowchart of a method of analyzing and displaying medical videos, according to some embodiments of the present disclosure.

FIG. 8 is a simplified diagram of a system for analyzing and displaying medical videos during an endoscopy procedure, according to some embodiments of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

As used herein, the term “network” may comprise any hardware or software-based framework that includes any artificial intelligence network or system, neural network or system and/or any training or learning models implemented thereon or therewith.

As used herein, the term “module” may comprise hardware or software-based framework that performs one or more functions. For example, a module may be implemented as one or more computer programs to be executed on programmable computers, each comprising some combination of at least one processor, a data storage system, at least one input device and/or at least one output device. In some embodiments, the module may be implemented on one or more neural networks.

Colonoscopy systems that can analyze videos from a wide field of view and that have an efficient and easy-to-use user interface can improve the detection of polyps, and therefore the diagnosis and treatment of colon cancers. Standard endoscopes for use in colonoscopies include one standard camera with a standard field of view. Because of the limited field of view, it may be difficult for physicians using standard endoscopes to view the entire colon, particularly within the folds of the colon. Systems have been proposed that use multiple cameras or one fisheye or wide-angle camera. Although these endoscopes may be able to collect video of a larger area of the colon, it may be difficult for the physician to effectively analyze the entire colon because there may be too much information for them to scan efficiently during a colonoscopy procedure. When multiple video feeds are shown on the screen, the physician may have trouble looking through all regions of the video while manipulating the endoscope. Moreover, fisheye cameras may be distorted around the periphery, making it difficult for physicians to identify polyps located around the periphery of the field of view.

The present invention overcomes these problems by leveraging the power of artificial intelligence (AI). Instead of having physicians analyze all of the videos collected, the secondary video from additional cameras as part of a multi-camera endoscope or the distorted portion of the video from the fisheye camera may be analyzed by a target feature detector. The target feature detector may analyze the secondary videos and detect the location of the polyp. The location of the polyp may be passed to an alert generator that generates an alert indicating the presence and/or location of the target feature detected. Thus, the primary video having a field of view similar to current endoscopy system may be displayed for the physician to visually analyze while the Al program analyzes the secondary videos. The AI will then alert the physician if a polyp is detected in the secondary video so the physician can decide to view the potential polyp.

Although some endoscopic systems of the present disclosure may be used for colonoscopies, other embodiments may be used in any procedure using endoscopes.

These descriptions are provided for example purposes only and should not be considered to limit the scope of the invention described herein. Certain features may be added, removed, or modified without departing from the spirit of the claimed subject matter.

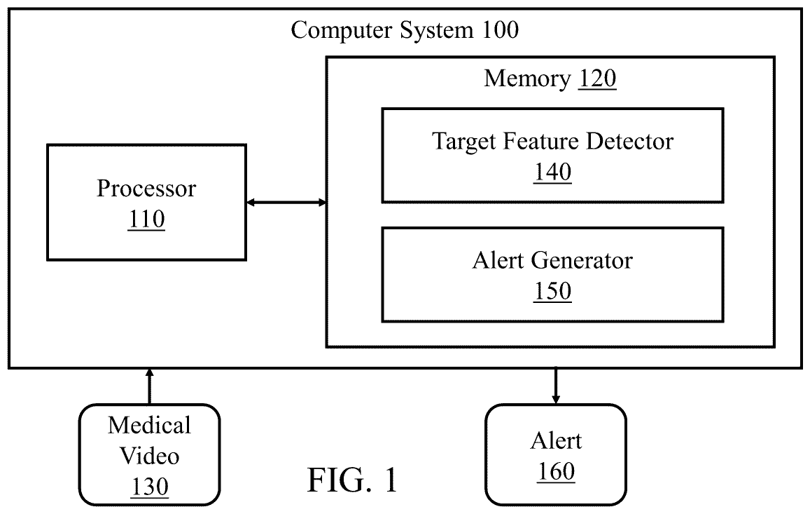

FIG. 1 is a schematic diagram illustrating a computer system 100 for implementing a target feature detector 140 and an alert generator 150, according to some embodiments of the present disclosure. The computer system 100 includes at least one processor 110 coupled to a memory 120. Although the computing device 100 is shown with only one processor 110, it is understood that processor 110 may be representative of one or more central processing units, multi-core processors, microprocessors, microcontrollers, digital signal processors, field programmable gate arrays (FPGAs), application specific integrated circuits (ASICs), graphics processing units (GPUs) and/or the like in the computing device 100. The computing device 100 may be implemented as a stand-alone subsystem, as a board added to a computing device, and/or as a virtual machine.

The memory 120 may be used to store software executed by computing device 100 and/or one or more data structures used during operation of computing device 100. The memory 120 may include one or more types of machine-readable media. Some common forms of machine-readable media may include floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor (e.g., the processor 110) or computer is adapted to read. In the present embodiments, for example, the memory 120 includes instructions suitable for training and/or using a target feature detector 140 and/or an alert generator 150 described herein.

The processor 110 and/or the memory 120 may be arranged in any suitable physical arrangement. In some embodiments, the processor 110 and/or the memory 120 are implemented on the same board, in the same package (e.g., system-in-package), on the same chip (e.g., system-on-chip), and/or the like. In some embodiments, the processor 110 and/or the memory 120 include distributed, virtualized, and/or containerized computing resources. Consistent with such embodiments, the processor 110 and/or the memory 120 may be located in one or more data centers and/or cloud computing facilities.

In some examples, the memory 120 may include non-transitory, tangible, machine readable media that includes executable code that when run by one or more processors (e.g., the processor 110) may cause the one or more processors to perform the methods described in further detail herein. For example, as shown, the memory 120 includes instructions for a target feature detector 140 and an alert generator 150 that may be used to implement and/or emulate the systems and models, and/or to implement any of the methods described further herein. In some embodiments, the target feature detector 140 may receive an input that includes a medical video 130 and may detect target features in the medical video 130. The medical video 130 may be collected from a camera system on an endoscope. The camera system may have an increased field of view compared to a standard endoscopic camera. In some embodiments, the camera system may include a primary camera and at least one secondary camera, where the medical video 130 is collected from the secondary camera or cameras. In other embodiments, the camera system is a fisheye camera with a larger field of view than a standard camera.

The target feature detector 140 may pass information about the target feature to the alert generator 150 including, for example, the number, location, size, severity, or other information about the target feature. In some cases, the medical video 130 or a modified medical video 130 may be passed along to the alert generator 150 as described in more detail below. The alert generator 150 may generate an alert 160 visually and/or auditorily indicating the presence of the target feature. The alert 160 may also include information about the target feature or images of the target feature.

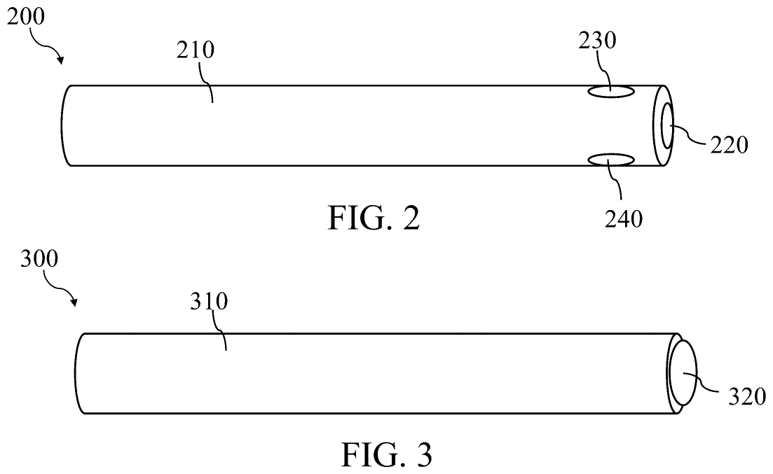

FIG. 2 is a simplified diagram of a multi-camera endoscope 200 according to some embodiments of the present disclosure. The multi-camera endoscope 200 may be used to collect medical videos 130. For example, the medical videos 130 may be collected during a colonoscopy, a laparoscopy, a cystoscopy, or any other endoscopic procedure. The multi-camera endoscope may include a catheter, sheath, sleeve, or body 210 and two or more cameras. The multi-camera endoscope 200 may comprise any appropriate number of cameras. For example, the multi-camera endoscope 200 may include 2, 3, 4, 5, 6, 7, 8, 9, or 10 cameras. In particular embodiments, including the illustrated embodiment, the multi-camera endoscope may include 3 cameras. There may be a primary camera 220 located at the tip of the body 210 such that the primary camera 220 faces forward and away from the body 210.

There may also be one or more secondary cameras 230, 240 located on the side of the body 210. The secondary cameras 230, 240 may be oriented such that the views of the secondary cameras 230, 240) are oriented perpendicularly to the view of the primary camera 220. In some embodiments, the views of the secondary cameras 230, 240 are angled towards or away from the view of the primary camera 220. The view of the secondary cameras 230, 240) may or may not overlap the view of the primary camera 220. The first secondary camera 230 may be directly opposite the second primary camera 240. In other cases, the first 230 and second 240) secondary cameras may be located at any circumferential position along the side of the body 210. The secondary cameras 230, 240 may be longitudinally aligned or staggered along the longitudinal surface of the body 210. The secondary cameras 230, 240 may be the same type of camera as the primary camera 220 or a different type of camera. For example, in some embodiments all cameras may be 4k resolution cameras. In other embodiments, the primary camera may be a 4k resolution camera and the secondary cameras may be 2k resolution cameras. In some embodiments, the primary camera 220 may have a standard field of view and the secondary cameras may be fisheye cameras with a wider field of view. The secondary cameras 230, 240 may be smaller than the primary camera 220. Together, the primary camera 220 and the secondary cameras 230, 240 may give the multi-camera endoscope 200 a larger field of view than a single standard camera field of view. For example, the field of view of the multi-camera endoscope may be 80, 100, 120, 140, 160, 180, 200, 220, 240, 260, 280, 300, 320, 340, or 360.

FIG. 3 is a simplified diagram of a fisheye camera endoscope 300 according to some embodiments of the present disclosure. The fish-eye camera endoscope 300 may include a catheter, sheath, sleeve, or body 310 and a fisheye or wide view camera 320. The fisheye or wide view camera 320 may have a field of view larger than the field of view of a standard camera. The fisheye camera 320 may be located at the tip of the body 310 such that the fisheye camera 320 faces forward and away from the body 310. The fisheye camera 320 may have any appropriate field of view. For example, the field of view of the fisheye camera 320 may be 80, 100, 120, 140, 160, 180, or 200. In some embodiments, the edges or periphery of the videos collected by the fisheye camera 320 may be distorted. The distortion of the video collected from the fisheye camera 320 may be increasingly distorted from the center of the video to the periphery.

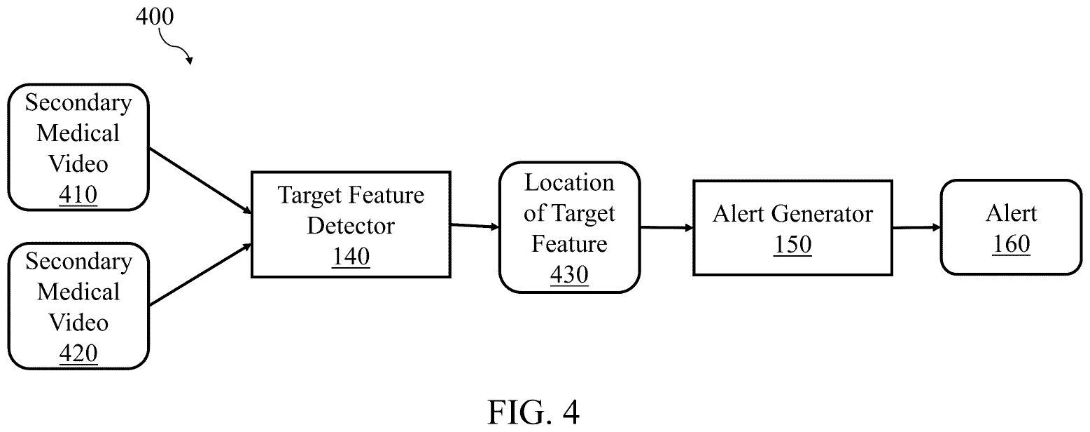

FIG. 4 is a block diagram 400 of a process for analyzing secondary medical videos 410, 420, according to some embodiments of the present disclosure. In the illustrated embodiment, a multi-camera endoscope 200 such as that described above in reference to FIG. 2 may be used to collect the medical videos 130. In these cases, the target feature detector 140) receives secondary medical videos 410, 420 collected from the secondary cameras 230, 240, respectively. In some embodiments, the video from the primary camera 220 is not input into the target feature detector 140. The target feature detector 140 may detect one or more target features in the secondary medical videos 410, 420. For example, the secondary medical videos 410, 420 are collected from a multi-camera endoscope 200 during a colonoscopy procedure and the target feature may be a polyp. The target feature detector 140 may detect the location 430 of the target feature. In some embodiments, the target feature detector 140 detects other information about the target feature including, for example, the number, size, or severity of the target feature.

Although a multi-camera endoscope is described in reference to FIG. 4, it is contemplated that the same or similar process can be used for analyzing medical video 130 collected from a fisheye camera endoscope 300 such as that described above in reference to FIG. 3. When a fisheye camera endoscope 300 is used, the periphery of the medical video 130 collected may be distorted and may be input into the target feature detector 140. In some embodiments, the center of the video collected by the fisheye camera 320 may not be input into the target feature detector 140.

The target feature detector 140 may use any appropriate computational structure that is configured to identify the presence and/or location of the target feature 430. The target feature detector 140 may include a neural network trained to identify the target feature.

In some embodiments, the target feature detector 140 may include a RetinaNet convolutional neural network (CNN) as described in Tsung-Yi Lin et al., Focal Loss for Dense Object Detection, arXiv: 1708.02002 (Feb. 7, 2018), the entirety of which is incorporated by reference herein. Each frame of the secondary medical videos 130 may be input into RetinaNet. RetinaNet may sample a large set of candidate object locations or anchor boxes across the frame at three scales and three aspect ratios for each location. A ResNet-50 network may be applied directly to the frame to extract the target features, which are then combined across multiple resolution levels. The target features extracted at each resolution level may then be fed into both a classification subnet and a box regression subnet. The classification subnet may predict the probability of the presence of the target feature for each anchor box across all positions. The classification subnet may be a convolutional network terminating in a convolutional filter with a number of filters equal to the number of anchors in each position multiplied by the number of object classes. The box regression subnet may be a fully convolutional network that predicts an offset from each anchor box to a nearby ground-truth object (if one exists). The box regression subnet may terminate in an output equal to four times the number of anchor boxes. The top predictions from each resolution level may be merged. Nonmaximum suppression with a threshold may be applied to the top predictions to yield a final prediction including the presence and/or location of the target feature 430.

In other embodiments, the target feature detector 140 may include a Long Short-Term Memory Single Shot Detector (LSTM-SSD) neural network as described in Mason Liu et al., Mobile Video Object Detection with Temporally-Aware Feature Maps, arXiv: 1711.06368v2 (Mar. 28, 2018), the entirety of which is incorporated by reference herein. LSTM-SSD may analyze previous frames to leverage better predictions for the current frame. The base of the LSTM-SSM may include a single shot multibox detector (SSD), which may be a single-frame detector similar to or the same as RetinaNet described above. All convolutional layers in the SSD may be depth-wise separable convolutions. In some cases, recurrent units may be operationally disposed between consecutive convolutional layers. The recurrent units may be ConvLSTM layers such that information from the current frame and previous frames may be analyzed. LSTM-SSD may output a prediction about the location of the target feature 430.

In some embodiments, the computational structure of the target feature detector 140) may include DEEP Detection of Elusive Polyps (DEEP²) as described in Dan M. Livovsky et al. Detection of elusive polyps using a large-scale artificial intelligence system (with videos). Clinical Endoscopy Vol. 94. No. 6. pg. 1099-1109 (2021), the entirety of which is incorporated by reference herein. As described. DEEP² may include any appropriate CNN capable of detecting target features. For example, in some embodiments. DEEP² may include RetinaNet, as described above, and, in other embodiments, may include LSTM-SSD, as described above. DEEP² may include a temporal logic layer that receives the prediction of the location of the target feature output from the CNN. The temporal logic layer may then refine the location of the target feature based on locations of the target feature detected in previous frames. The previous n frames including the current frame may be analyzed. If a certain threshold of the n frames have at least one detection, then the location of the target feature 430) detected in the current frame is confirmed and output from DEEP².

In some embodiments, the target feature detector 140 may be trained independent of the alert generator 150. In some embodiments, the training data for the target feature detector 140 may include medical videos with the location of the target feature detector labelled. In some embodiments, the location of the target feature is labelled by a medical professional. For example, the medical video may be collected during a colonoscopy and the target feature may be a polyp. In this case, the medical professional may be a gastroenterologist who labels the location of polyps in the medical video. The target feature detector 140 may be trained to minimize the loss associated with the detection of the location of the target features. In some embodiments, the target feature detector 140 may be trained using standard stochastic gradient descent or any other appropriate method. A validation set of the training data may be used to validate the accuracy of the trained target feature detector 140.

The alert generator 150 may receive the location of the target feature 430 or any other information about the target feature from the target feature detector 140. In some embodiments, the alert generator 150 may generate an alert 160, which can be passed along to a display to notify the physician that a target feature has been detected in one or more of the secondary medical videos 410, 420. In some cases, the alert 160 may simply indicate that a target feature was identified without indicating where the target feature was located. The physician may then move the multi-camera endoscope 200 so that the primary camera 220 can scan the area imaged by the secondary cameras 230, 240 so that the physician can visually identify and confirm the existence and location of the target feature. In other embodiments, the physician may toggle from the primary camera 220 to one or more of the secondary cameras 230, 240 to view the target feature. The physician may visually identify other characteristics of the target feature that may be important to determining whether additional action needs to be taken in response to the target feature. For example, in some embodiments, the multi-camera endoscope 200 may collect medical videos 130 during a colonoscopy procedure. Thus, the alert 160 may identify a polyp along the walls of the colon. When the physician receives the alert 160, the physician may decide to note the existence and characteristics of the polyp for further monitoring or may remove part or all of the polyp. In addition to a visual alert, the alert 160 may also include an auditory alert such as a buzzer, beep, tone, or other warning indicating a target feature has been detected.

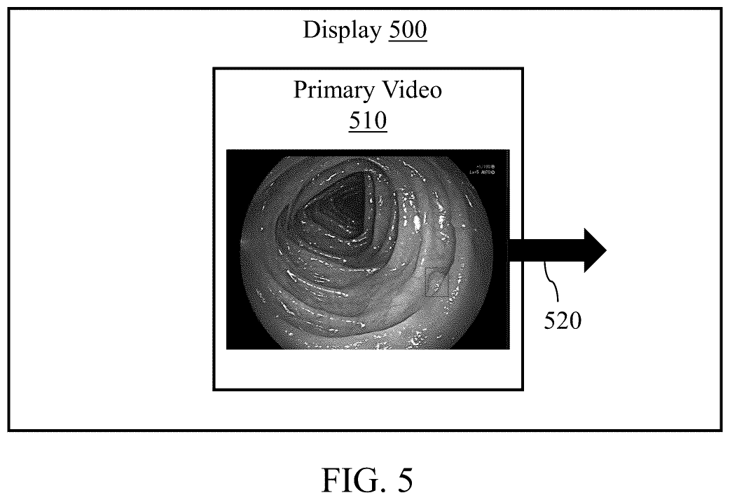

In some embodiments, the alert 160 may not only alert the physician that a target feature was detected, but it may also indicate the location of the target feature 430. FIG. 5 illustrates an example display 500 in which the alert 520 indicates the location of the target feature 430 relative to the primary video 510, according to some embodiments of the present disclosure. The display 500 includes the primary video 510 collected from a primary camera. When a target feature is detected by the target feature detector 140, the alert generator 150 generates an alert 520, which is displayed on display 500. In the illustrated example, the alert 520 indicates the location of the target feature 430. In some embodiments, the alert generator 150 may also receive information about the location and field of view of the primary video 510. The alert generator 150 may analyze the location and field of view of the primary video 510 and the secondary video to determine the location of the target feature 430 in the secondary video relative to the primary video 510. In some embodiments, the relative location of the primary and secondary videos may be known. In some embodiments, the relative location of the primary and secondary videos may be measured in any appropriate way. In the illustrated embodiment, the alert 520 is pointed towards the right, indicating that the target feature has been detected in the secondary videos and is located to the right of the primary video 510.

In the illustrated embodiment, the alert 520 may be an arrow that points to the location of the target feature. In other embodiments, the alert 520 may be a triangle, line, or any other appropriate indicator illustrating the location of the target feature. Moreover, the alert 520 may be any appropriate color. For example, the alert 520 may be black, green, red, yellow, blue, or orange. The alert may also include more than one color. In some embodiments, the alert 520 is an outline with a white or unfilled center. The alert 520 may also include a message with text describing the nature of the alert and/or the location of the target feature.

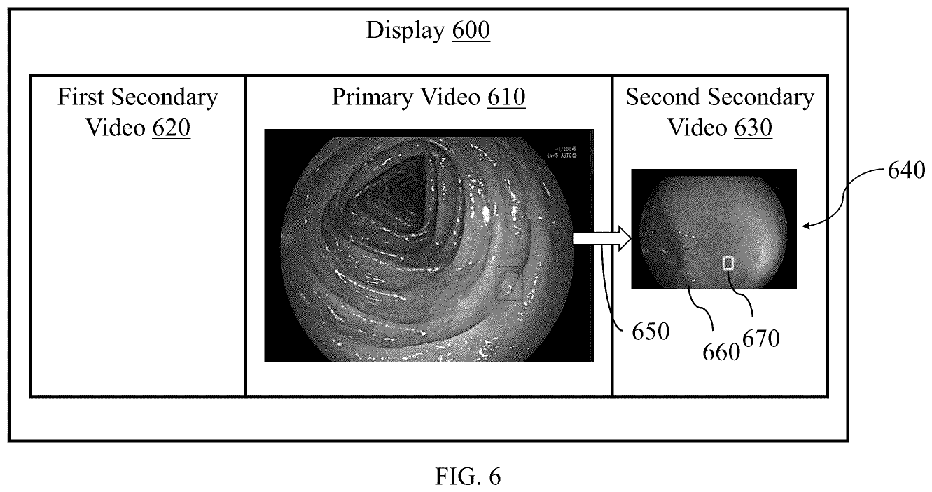

In some embodiments, the alert 160 generated by the alert generator 150 may also include part of the secondary video including the target feature. FIG. 6 illustrates an example display 600 where the alert 640 includes a part of the secondary video 660, according to some embodiments of the present disclosure. The display 600 may include the primary video 610 and space for the first secondary video 620 and the second secondary video 630. When a target feature is detected in one of the secondary videos 620, 630, the alert generator 150 generates an alert 640. The alert 640 may include an arrow or other shape 650 indicating the location of the target feature 430 as described above in reference to FIG. 5. Moreover, the alert 640 may also include a part of the secondary video 660 where the target feature was detected. In some cases, the secondary video 660 may be a still image including the target feature. In other cases, the secondary video 660 may be a video that may have a length of any amount of time. For example, the secondary video 660 may be a video in a range of a few seconds long to several minutes long. The secondary video 660 may also be a live video from the secondary camera.

In some embodiments, the location of the target feature 430 may be bounded on the part of the secondary video 660 by a box 670. The box 670 may be any appropriate shape including, for example, a square, rectangle, circle, oval, hexagon, or any other appropriate shape. The box 670 may be any appropriate color including, for example, black, green, red, yellow, blue, or orange.

In this example, the secondary video 660 is from the second secondary video 630. The alert 640 includes part of the secondary video 660 shown in the area for the secondary video 660 on the display 600. There is an arrow 650 that points from the primary video 610 towards the part of the secondary video 660. A box 670 encircles the location of the target feature 430. Thus, the physician can easily view the target feature.

Although a multi-camera endoscope is described in reference to FIGS. 4-6, it is contemplated that the same or similar displays can be used for analyzing medical video 130 collected from a fisheye camera endoscope 300 such as that described above in reference to FIG. 3. When a fisheye camera endoscope 300 is used, the periphery of the medical video 130 collected may be distorted and may be input into the target feature detector 140. Thus, the alert 160 may indicate the presence and/or the location of the target feature in the periphery of the medical video 130. The alert 160 may also include a part of the peripheral video that includes the target feature, as described above.

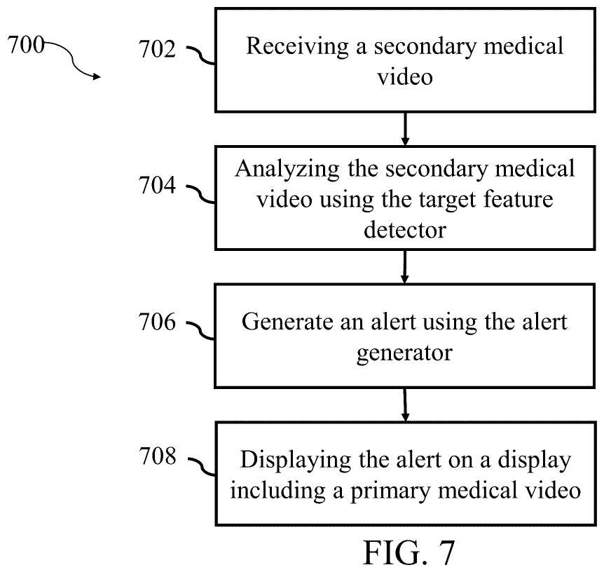

FIG. 7 is a flowchart of a method 700 of using a target feature detector 140 and alert generator 150 to identify target features in medical videos 130 and alert in response to the identification, according to some embodiments of the present disclosure. Step 702 includes receiving a secondary medical video. In some embodiments, the secondary medical videos may be received from multiple cameras. For example, there may be a first secondary camera 230 and a second secondary camera 240 that collect first secondary medical video 410 and second secondary medical video 420, respectively, and pass the videos 410, 420 along to the target feature detector 140. In some embodiments, the cameras 230, 240 may be located on a multi-camera endoscope 200, which may be used to collect video during a colonoscopy procedure.

Step 704 may include analyzing the secondary medical videos using the target feature detector 140. As described in more detail above, the target feature detector 140 may include any appropriate computational structure to identify the location of the target feature 430. For example, the target feature detector 140 may include a DEEP² architecture which uses a RetinaNet or LSTM-SSD CNN. The location of the target feature 430 may include a region encompassing the target feature, coordinates, or any other appropriate information. The target feature detector 140 may also output any other relevant information about the target feature.

Step 706 may include generating an alert 160 using the alert generator 150. As described in more detail above, the alert 160 may indicate the presence and/or location of the target feature. Generating the alert 160 may include determining the location of the primary video relative to the location of the secondary medical video and the target feature, as described in more detail below.

Step 708 may include displaying the alert on a display including a primary medical video. The display may be in communication with a processor implementing the target feature detector and the alert generator. In some embodiments, the display is also in communication with the multi-camera endoscope 200 and receives the primary and/or secondary videos therefrom. The display may be any appropriate display including those described in reference to FIGS. 5 and 6 above. The alert 160 displayed may include an arrow 520, 650 or any other appropriate shape pointing to the location of the target feature 430. In some embodiments, the alert 160 also includes a part of the secondary video 660 including the target feature. The location of the target feature 430 in the part of the secondary video 660 may be bounded by a box 670. In some embodiments, the alert 160 may also include an auditory signal.

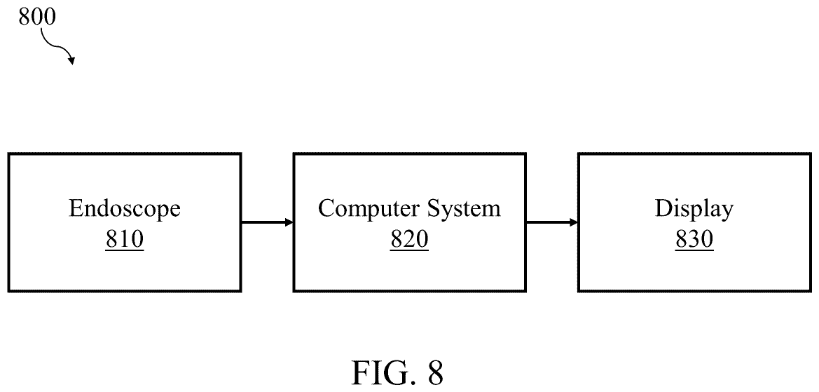

A multi-camera endoscope 200 may capture a video signal from each camera 220, 230, 240 and communicate the video signals to a computer system for processing, such as computer system 100. The computer system 100 may be remote from the multi-camera endoscope 200. For example, the computer system 100 and multi-camera endoscope 200 may be connected via any wired or wireless communication connection, such as via the Internet or via a WiFi network. Alternatively, the computer system 100 and multi-camera endoscope 200 may be part of the same system or apparatus and located together in a room where endoscopy procedures are performed, and the multi-camera endoscope 200 may be connected to the computer system 100 via a conductive wire or cable. Additionally, a display, such as displays 500 or 600, may be connected to computer system 100 via any wired or wireless connection, such as via a directly wired cable, via the Internet or via a WiFi network, so that the display may receive one or more signals to be displayed from computer system 100. FIG. 8 illustrates an example of a system 800 for analyzing and displaying medical videos during an endoscopy procedure. The system includes an endoscope 810, a computer system 820, and a display 830. The endoscope 810 may be a multi-camera endoscope, such as endoscope 200, or a fisheye camera endoscope 300. An example of computer system 820 is as shown in FIG. 1, and an example of display 830 is as shown in FIG. 5 or 6.

A number of variations are possible on the examples and embodiments described above. Accordingly, the logical operations making up the embodiments of the technology described herein are referred to variously as operations, steps, objects, elements, components, layers, modules, or otherwise. Furthermore, it should be understood that these may occur in any order, unless explicitly claimed otherwise or a specific order is inherently necessitated by the claim language.

Generally, any creation, storage, processing, and/or exchange of user data associated with the method, apparatus, and/or system disclosed herein is configured to comply with a variety of privacy settings and security protocols and prevailing data regulations, consistent with treating confidentiality and integrity of user data as an important matter. For example, the apparatus and/or the system may include a module that implements information security controls to comply with a number of standards and/or other agreements. In some embodiments, the module receives a privacy setting selection from the user and implements controls to comply with the selected privacy setting. In some embodiments, the module identifies data that is considered sensitive, encrypts data according to any appropriate and well-known method in the art, replaces sensitive data with codes to pseudonymize the data, and otherwise ensures compliance with selected privacy settings and data security requirements and regulations.

In several example embodiments, the elements and teachings of the various illustrative example embodiments may be combined in whole or in part in some or all of the illustrative example embodiments. In addition, one or more of the elements and teachings of the various illustrative example embodiments may be omitted, at least in part, and/or combined, at least in part, with one or more of the other elements and teachings of the various illustrative embodiments.

Any spatial references such as, for example, “upper,” “lower,” “above,” “below,” “between,” “bottom,” “vertical,” “horizontal,” “angular,” “upwards,” “downwards,” “side-to-side,” “left-to-right,” “right-to-left,” “top-to-bottom,” “bottom-to-top,” “top,” “bottom,” “bottom-up,” “top-down,” etc., are for the purpose of illustration only and do not limit the specific orientation or location of the structure described above. Connection references, such as “attached,” “coupled,” “connected,” and “joined” are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” Unless otherwise noted in the claims, stated values shall be interpreted as illustrative only and shall not be taken to be limiting.

Additionally, the phrase “at least one of A and B” should be understood to mean “A, B, or both A and B.” The phrase “one or more of the following: A, B, and C” should be understood to mean “A, B, C, A and B, B and C, A and C, or all three of A, B, and C.” The phrase “one or more of A, B, and C” should be understood to mean “A, B, C, A and B, B and C, A and C, or all three of A, B, and C.”

Although several example embodiments have been described in detail above, the embodiments described are examples only and are not limiting, and those skilled in the art will readily appreciate that many other modifications, changes, and/or substitutions are possible in the example embodiments without materially departing from the novel teachings and advantages of the present disclosure. Accordingly, all such modifications, changes, and/or substitutions are intended to be included within the scope of this disclosure as defined in the following claims.

What is claimed is:

1. A method of analyzing and displaying medical videos during an endoscopy procedure using a multi-camera endoscope having a first camera and a second camera, comprising:
receiving a first medical video from a first camera;
receiving a second medical video from a second camera;
detecting one or more target features of the second medical video using a pre-trained target feature detector comprising a neural network;
generating an alert indicating the detection of the one or more target features in the second medical video; and
displaying the first medical video and the alert on a display.

2. The method of claim 1, wherein the endoscopy procedure is a colonoscopy and wherein the one or more target features comprises a polyp.

3. The method of claim 1, wherein the first medical video comprises a first field of view and the second medical video comprises a second field of view.

4. The method of claim 3, wherein the first field of view is different than the second field of view.

5. The method of claim 4, further comprising determining a location of the second field of view relative to the first field of view.

6. The method of claim 5, wherein detecting one or more target features comprises detecting a location of the one or more target features in the second field of view.

7. The method of claim 6, wherein the alert indicates a location of the one or more target features relative to the first field of view.

8. The method of claim 1, wherein the alert comprises a part of the second medical video comprising the one or more target features.

9. The method of claim 8, wherein the alert comprises a box encompassing one or more of the one or more target features.

10. The method of claim 1, wherein the first camera and the second camera are coupled to an endoscope.

11. The method of claim 1, wherein the target feature detector comprises a domain-specific pre-trained machine learning model.

12. A system for analyzing and displaying medical videos during an endoscopy procedure, comprising:
an endoscope comprising a first camera and a second camera;
a display; and,
a processor configured to:
receive a first medical video from the first camera;
receive a second medical video from the second camera;
detect one or more target features of the second medical video using a target feature detector;
generate an alert indicating the detection of the one or more target features in the second medical video; and
display the first medical video and the alert on the display.

13. The system of claim 12, wherein the first medical video comprises a first field of view and the second medical video comprises a second field of view.

14. The system of claim 13, wherein the first field of view is different than the second field of view.

15. The system of claim 14, wherein the processor is further configured to determine a location of the second field of view relative to the first field of view.

16. The system of claim 15, wherein the processor is further configured to detect a location of the one or more target features in the second field of view.

17. The system of claim 16, wherein the alert indicates a location of the one or more target features relative to the first field of view.

18. A system for analyzing and displaying medical videos during an endoscopy procedure, comprising:
a first camera configured to receive a first medical video comprising a first field of view;
a second camera configured to receive a second medical video comprising a second field of view different from the first field of view;
a display configured to display at least the first medical video;
a processor configured to:
receive the second medical video;
detect a target feature of the second medical video using a pre-trained target feature detector comprising a neural network;
generate an alert indicating the detection of the target feature in the second medical video; and
display the alert on the display.

19. The system of claim 18, wherein the alert is displayed adjacent to the first medical video.

20. The system of claim 18, wherein the alert comprises a part of the second medical video comprising the target feature.
